(19) United States
(12) Patent Application Publication
(10) Pub. No.: US 2011/0238137 A1
(43) Pub. Date: Sep. 29, 2011

(54) Medical apparatus for photodynamic therapy and method for controlling therapeutic light

(75) Inventor: Kazuhiro Tsujita, Kanagawa (JP)

(73) Assignee: FUJIFILM Corporation, Tokyo (JP)

(21) Appl. No.: 12/929,475

(22) Filed: Jan. 27, 2011

(30) Foreign Application Priority Data
Mar. 25, 2010 (JP) 2010-069314

(51) Int. Cl. A61N 5/06

(52) U.S. Cl. 607/88

(57) ABSTRACT

A laser source irradiates a tumor site, permeated with photosensitizer, using pulse laser for PAI. The photosensitizer absorbs the pulse laser for PAI to generate ultrasonic waves. An ultrasonic wave detection device detects the ultrasonic waves to output an ultrasonic signal. A processor device performs various processes to the ultrasonic signal to generate image data. Based on the image data, a photosensitizer concentration calculator calculates concentration distribution of the photosensitizer at a specific depth in the tumor site. Based the obtained concentration distribution, an irradiation condition of CW laser for photodynamic therapy (PDT) is set or changed. Based on the irradiation condition, the CW laser for PDT is irradiated to the tumor site.

### FIELD OF THE INVENTION

The present invention relates to a medical apparatus for photodynamic therapy and a method for controlling therapeutic light.

### BACKGROUND OF THE INVENTION

Recently, photodynamic diagnosis (PDD) and photodynamic therapy (PDT), both using laser, have been rapidly progressing along with the development of electronic medical technology. Before performing the PDD, a photosensitizer, for example, hematoporphyrin derivative, reacting to light in a specific wavelength range is administered to a patient to accumulate or permeate the photosensitizer into the patient's tumor site. Thereafter, light in a first wavelength range (for example, 405 nm) is irradiated to the tumor site. Thereby, the photosensitizer accumulated in the tumor site reacts to the light to generate fluorescence. Thereby, it becomes easy to discriminate the tumor site from the normal site, allowing an operator or doctor to find the tumor site through visual inspection. After locating the tumor site using the PDD, the operator or doctor performs the PDT. For the PDT, therapeutic light in a second wavelength range (for example, 630 nm) is irradiated to the tumor site where the photosensitizer has been accumulated. Thereby, the photosensitizer is activated, causing necrosis of the tumor site.

The therapeutic light used for the PDT causes the photosensitizer to generate reactive oxygen species to kill the tumor site. Accordingly, an amount of the therapeutic light irradiated is extremely large compared to that in the PDD. However, when the tumor site is in the course of necrosis, the therapeutic light having a large light amount may cause damage to a normal site surrounding the tumor site. To solve this problem, in U.S. Patent Application Publication No. 2008/0221647, pulsed light is irradiated to the tumor site to generate ultrasonic waves in the tumor site. The feedback control using a signal of the ultrasonic waves allows the therapeutic light irradiation with an appropriate light amount. A method to generate ultrasonic waves in living tissue using pulsed light irradiation to obtain ultrasonic tomographic information is referred to as photoacoustic spectroscopy in U.S. Pat. No. 6,979,292 corresponding to Japanese Patent Laid-Open Publication No. 2005-21380, for example.

Prior to the PDT, the photosensitizer administered to a patient not only accumulates at the surface of the tumor site but also inside to the bottom of the tumor site. Irradiation of the therapeutic light to the tumor site gradually reduces the concentration of the photosensitizer at the surface of and inside the tumor site. During the PDT, the decrease in the concentration of the photosensitizer at the surface of the tumor site is easily recognized through visual inspection or with the use of an image of the tumor site. On the other hand, it is difficult to keep track of the decrease in the concentration of the photosensitizer inside the tumor site. An insufficient amount of therapeutic light applied to the inside of the tumor site results in an insufficient therapeutic effect. Conversely, when an excessive amount of therapeutic light is irradiated to the inside of the tumor site to obtain a sufficient therapeutic effect, it may cause complications such as perforation.

To solve the above problem, the technique of U.S. Patent Application Publication No. 2008/0221647 may be used. However, in U.S. Patent Application Publication No. 2008/0221647, the ultrasonic signal received by an ultrasonic wave detector is used for the feedback control with no consideration given to the concentration distribution of the photosensitizer at the surface or at a specific depth in the tumor site. Thus, this technique cannot solve the above-described problem.

### SUMMARY OF THE INVENTION

An object of the present invention is to provide a medical apparatus for photodynamic therapy (PDT) and a method for controlling therapeutic light for properly irradiating a tumor site, into which photosensitizer is permeated, using therapeutic light based on the concentration distribution of the photosensitizer at a specific depth in the tumor site.

In order to achieve the above and other objects, a medical apparatus for PDT includes a light source, an ultrasonic wave detection device, a concentration distribution calculator, and a therapeutic light controller. The light source irradiates concentration measurement light and therapeutic light to a tumor site. The concentration measurement light is used for obtaining concentration of a photosensitizer permeated into the tumor site. The therapeutic light is used for treating the tumor site. Alight amount of each of the concentration measurement light and the therapeutic light is adjustable. The ultrasonic wave detection device detects ultrasonic waves to output an ultrasonic signal. The ultrasonic waves are generated by the photosensitizer absorbing the concentration measurement light. The concentration distribution calculator obtains concentration distribution of the photosensitizer at a specific depth in the tumor site based on the ultrasonic signal. The therapeutic light controller controls an irradiation condition of the therapeutic light to be irradiated to the tumor site based on the concentration distribution.

It is preferable that a portion having signal intensity equal to or higher than a predetermined value in the tumor site is set as a region of interest, and the irradiation condition of the therapeutic light is controlled such that the speed in reducing the concentration differs according to the concentration distribution.

It is preferable that the irradiation condition is controlled such that the speed in reducing the concentration increases at a predetermined rate in a portion having the photosensitizer in high concentration in the region of interest, and the speed in reducing the concentration decreases at a predetermined rate in a portion having the photosensitizer in low concentration in the region of interest.

It is preferable that the irradiation condition is controlled such that the speed in reducing the concentration decreases at a predetermined rate in a portion having the photosensitizer in high concentration in the region of interest, and the speed in reducing the concentration increases at a predetermined rate in a portion having the photosensitizer in low concentration in the region of interest.

It is preferable that the medical apparatus for PDT further includes a PDT-index calculator for obtaining a PDT-index based on a concentration distribution of the photosensitizer at the specific depth obtained after the last irradiation of the therapeutic light and power of the irradiated therapeutic light. The PDT-index represents a therapeutic effect of irradiating the tumor site with the therapeutic light.

It is preferable that the medical apparatus for PDT further includes an irradiation time calculator for calculating, based on the PDT-index, irradiation time of the therapeutic light required for completion of therapy of the tumor site.

It is preferable that every time the concentration distribution is obtained, the obtained concentration distribution is displayed in a form of an image on a monitor.

It is preferable that the medical apparatus for PDT further includes a distribution matching section for matching the irradiation distribution of the concentration measurement light to the irradiation distribution of the therapeutic light.

It is preferable that the therapeutic light is continuously irradiated in a predetermined light amount from the light source.

It is preferable that the therapeutic light is irradiated from the light source while the light amount of the therapeutic light is changed.

It is preferable that a part of the therapeutic light is used as the concentration measurement light.

A method for controlling therapeutic light according to the present invention includes an irradiation step, a detection step, an obtaining step, and a controlling step. In the irradiation step, a tumor site is irradiated with concentration measurement light for obtaining concentration of a photosensitizer permeated into the tumor site. A light amount of the concentration measurement light is adjustable. In the detection step, ultrasonic waves are detected to output an ultrasonic signal. The ultrasonic waves are generated by the photosensitizer absorbing the concentration measurement light. In the obtaining step, a concentration distribution of the photosensitizer is obtained at a specific depth in the tumor site based on the ultrasonic signal. In the controlling step, an irradiation condition of the therapeutic light is controlled based on the concentration distribution.

It is preferable that in the controlling step, the irradiation condition of the therapeutic light is controlled such that a speed in reducing the concentration differs according to the concentration distribution.

It is preferable that the method further includes a power detecting step, a repeating step, and a PDT-index obtaining step. In the power detecting step, the power of the therapeutic light irradiated based on the irradiation condition is detected. In the repeating step, the an irradiation step, a detection step, and an obtaining step are repeated after the irradiation of the therapeutic light to obtain an updated concentration distribution of the photosensitizer at the specific depth in the tumor site. In the PDT-index obtaining step, a PDT-index is obtained based on the updated concentration distribution and the detected power of the therapeutic light. The PDT index represents a therapeutic effect of irradiating the tumor site with the therapeutic light.

According to the present invention, the tumor site into which the photosensitizer is permeated is irradiated with the concentration measurement light. The photosensitizer absorbs the concentration measurement light to generate ultrasonic waves. The ultrasonic waves are detected to output the ultrasonic signal. Based on the ultrasonic signal, the concentration distribution of the photosensitizer at the specific depth in the tumor site is obtained. Based on the concentration distribution, the irradiation condition of the therapeutic light is controlled. Thus, the therapeutic light is properly irradiated to the specific depth in the tumor site.

### BRIEF DESCRIPTION OF THE DRAWINGS

The above and other objects and advantages of the present invention will be more apparent from the following detailed description of the preferred embodiments when read in connection with the accompanied drawings, wherein like reference numerals designate like or corresponding parts throughout the several views, and wherein:

FIG. 1 is a schematic view of a medical apparatus for photodynamic therapy according to the present invention;

FIG. 2 is a graph showing relations between amplitude values PA of ultrasonic signals and absorbance A at different depths;

FIG. 3 is a graph showing the amplitude values PA of the ultrasonic signal at a specific depth;

FIG. 4 is a flow chart showing an operation of the present invention;

FIG. 5A is a waveform chart showing pulse laser for the PDT; and

FIG. 5B is a waveform chart showing that a part of the pulse laser for PDT shown in FIG. 5A is used as laser for PAI.

### DESCRIPTION OF THE PREFERRED EMBODIMENTS

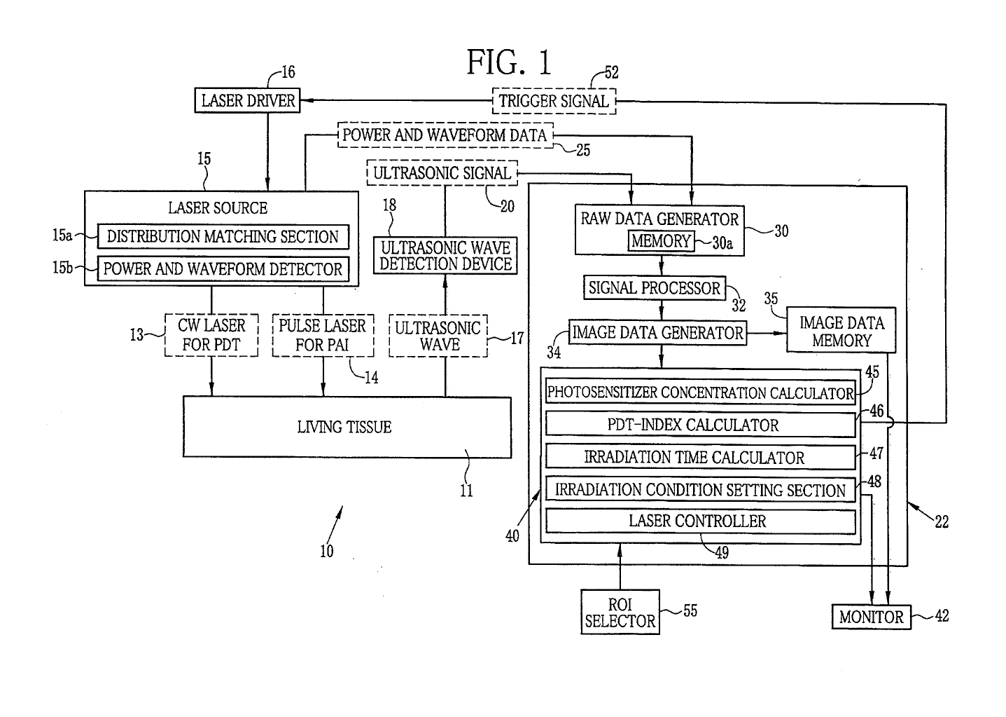

As shown in FIG. 1, a medical apparatus 10 or therapy apparatus for photodynamic therapy (hereafter abbreviated as PDT) of the present invention irradiates a tumor site, permeated with a photosensitizer or photosensitive agent, using light for PDT in a specific wavelength range. Thereby, pharmacological action of drug or the activated or excited photosensitizer treats or kills the tumor site. The medical apparatus 10 creates an image of the tumor site being treated with the use of PAI (photo acoustic imaging). The medical apparatus 10 is provided with a laser source 15, an ultrasonic wave detection device 18, and a processor device 22. The laser source 15 irradiates CW (Continuous Wave) laser 13 for PDT and pulse laser 14 for PAI to living tissue 11 having the tumor site. The ultrasonic wave detection device 18 detects ultrasonic waves 17 generated in the living tissue 11 by the irradiation of the pulse laser 14 for PAI to output an ultrasonic signal 20. The processor device 22 generates image data based on the ultrasonic signal 20 detected by the ultrasonic wave detection device 18 and then obtains concentration of the photosensitizer at a specific depth in the tumor site using the image data. Examples of the processor devices 22 include a PC (Personal Computer), a DSP (Digital Signal Processor), and an FPGA (Field Programmable Gate Array).

The laser source 15 is a laser diode or a solid-state laser, for example. Under the control of a laser driver 16, the laser source 15 irradiates the CW laser 13 for PDT to treat or kill the tumor site into which the photosensitizer is permeated. The CW laser 13 for PDT is light in a specific wavelength range continuously irradiated in a predetermined or constant light amount or while a light amount is changed. The light amount of the CW laser 13 for PDT is adjustable. The irradiation of the CW laser 13 for PDT to the tumor site causes the photosensitizer permeated into the tumor site to produce reactive oxygen species, thereby resulting in necrosis or the like of the tumor site. Thus, the tumor site is treated.

The laser source 15 irradiates the tumor site with the pulse laser 14 for PAI to obtain an ultrasonic tomographic image of the tumor site and concentration information of the photosensitizer permeated into the tumor site. The pulse laser 14 for PAI is the light having a specific wavelength range. A light amount of the pulse laser 14 for PAI is changeable or adjustable. The photosensitizer permeated into the tumor site absorbs the pulse laser 14 for PAI irradiated to the tumor site. Thereby, the photosensitizer generates ultrasonic waves 17. The ultrasonic wave detection device 18 detects the ultrasonic waves 17. Absorption wavelengths of the photosensitizer are around 510 nm, 545 nm, 580 nm, and 630 nm when protoporphyrin, laserphyrin, or hematoporphyrin is used.

Before the irradiation of the pulse laser 14 for PAI and the CW laser 13 for PDT from the laser source 15, a distribution matching section 15a adjusts the irradiation distribution of the CW laser 13 for PDT and the irradiation distribution of the pulse laser 14 for PAI to match or coincide with each other. A power and waveform detector 15b detects power and waveform of each of the CW laser 13 for PDT and the pulse laser 14 for PAI, irradiated to the living tissue 11, as power and waveform data 25. The power and waveform data 25 is sent to the processor device 22 and used for generating one frame of RAW data.

The ultrasonic wave detection device 18 is provided with a plurality of acoustic transducer elements arranged in an array. The acoustic transducer elements convert the ultrasonic waves 17 from the photosensitizer into the ultrasonic signal 20 (may be referred to as RF signal). The ultrasonic signal 20 is sent to a RAW data generator 30 in the processor device 22.

The RAW data generator 30 generates one frame of RAW data based on the power and waveform data 25 of the pulse laser 14 for PAI detected by the power and waveform detector 15b and the ultrasonic signal 20 detected by the ultrasonic wave detection device 18. Every time one frame of RAW data is generated, the RAW data generator 30 stores the generated RAW data in a memory 30a. A signal processor 32 performs various processes such as a noise reduction process to the RAW data stored in the memory 30a. Thereafter, the RAW data is sent to an image data generator 34.

Based on the processed RAW data, the image data generator 34 generates image data of an ultrasonic tomographic image of the tumor site in the living tissue 11. The image data is stored in an image data memory 35 and sent to a controller 40 in the processor device 22. When the image data is stored in the image data memory 35, the ultrasonic tomographic image of the tumor site in the living tissue 11 is displayed on a monitor 42 based on the image data.

The controller 40 is provided with a photosensitizer concentration calculator 45, a PDT-index calculator 46, an irradiation time calculator 47, an irradiation condition setting section 48, and a laser controller 49. With the use of the image data, the photosensitizer concentration calculator 45 calculates or obtains concentration distribution of the photosensitizer located at a specific depth in the tumor site. The PDT-index calculator 46 calculates or obtains a PDT-index based on the obtained concentration of the photosensitizer. The PDT-index represents a therapeutic effect (hereafter may referred to as PDT effect) of irradiating the tumor site with the therapeutic light or the PDT (photodynamic therapy). Based on the PDT-index, the irradiation time calculator 47 calculates or obtains an irradiation time of the CW laser for the PDT required for the therapy. The irradiation condition setting section 48 sets or changes an irradiation condition of the CW laser 13 for PDT based on the concentration distribution of the photosensitizer obtained by the photosensitizer concentration calculator 45. The laser controller 49 generates a trigger signal 52 based on the irradiation condition set or changed by the irradiation condition setting section 48 and controls the laser driver 16 based on the trigger signal 52.

The photosensitizer concentration calculator 45 calculates or obtains a concentration distribution of the photosensitizer located at a specific depth D in the tumor site in a region of interest (hereafter referred to as ROI). The ROI is a portion with the signal intensity above a predetermined value from among the portions within the tumor site from which the photosensitizer emits light. Using an ROI selector 55, an operator or doctor specifies or selects the ROI prior to or in accordance with the therapy. The photosensitizer concentration calculator 45 specifies measurement positions P1 to Pn in the ROI at the specific depth D in the tumor site and then obtains amplitude values PA_{P1} to PA_{Pn} of the ultrasonic signals 20 at the measurement positions P1 to Pn, respectively. The photosensitizer concentration calculator 45 specifies or obtains optical path lengths L_{P1} to Lₚₙ (cm) between the measurement positions P1 to Pn and an exit section of the laser source 15, respectively. Here, “n” is a natural number equal to or larger than 2. Alternatively or in addition, the photosensitizer concentration calculator 45 may obtain the concentration distribution of the entire tumor site.

Using the following mathematical expressions (1) and (2), based on the amplitude values PA_{P1} to PA_{Pn} and the optical path lengths L_{P1} to Lₚₙ, concentrations C_{P1} to C_{Pn} (mM) of the photosensitizer in the measurement positions P1 to Pn at the specific depth D are obtained. Thus, the concentration distribution of the photosensitizer at the specific depth D is obtained. Every time a concentration distribution is obtained, the concentration distribution displayed on the monitor 42 is updated, allowing the operator to check the reduction of the concentration in the ROI as color fading on the monitor 42.

PA=a×A^{b} (1)

A=2.3×εm×L×C (2)

Here, A (cm⁻¹) denotes the absorbance in a position at the specific depth D. In the above mathematical expressions (1) and (2), for the sake of simplicity, numerical subscripts P1 to Pn for “PA”, “A”, “L”, and “C” are omitted. Each of “a”, “b”, “εm” is a constant. Each of “a” and “b” is a positive number which changes in accordance with the depth D. “εm” is a coefficient specific to the photosensitizer.

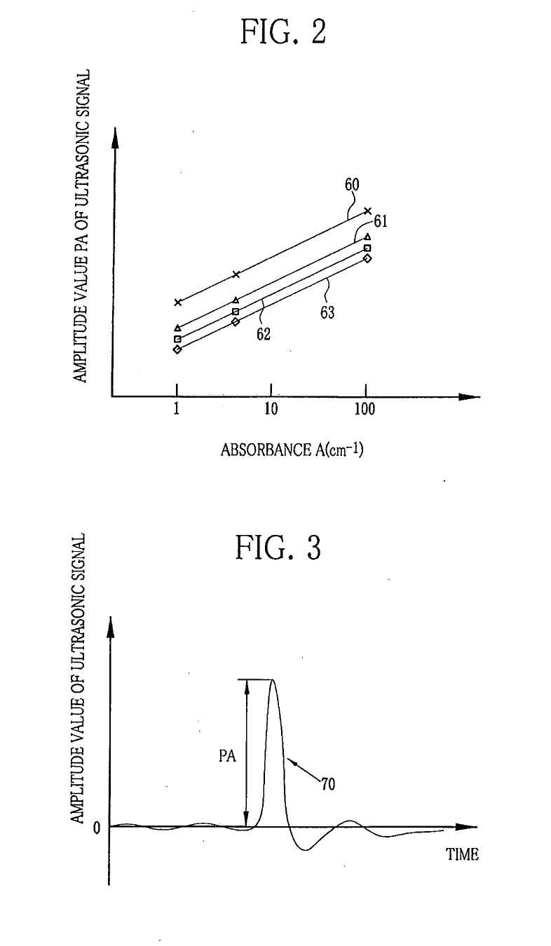

For example, as shown in FIG. 2, a relation between the absorbance A and the amplitude value PA of the ultrasonic signal 20 changes in accordance with the depth D. In FIG. 2, a line 60 denotes the relation between the absorbance A and the amplitude value P when the depth D is 5 mm. A line 61 denotes the relation between the absorbance A and the amplitude value P when the depth D is 10 mm. A line 62 denotes the relation between the absorbance A and the amplitude value P when the depth D is 15 mm. A line 63 denotes the relation between the absorbance A and the amplitude value P when the depth D is 20 mm. As shown in FIG. 2, at the same absorbance value, the shallower the depth D, the larger the amplitude value PA of the ultrasonic signal 20. The ultrasonic signal 20 at a specific depth is shown in FIG. 3. In this embodiment, the amplitude value PA of the ultrasonic signal 20 denotes a positive portion of a peak amplitude (peak portion) 70 of the ultrasonic signal 20 shown in FIG. 3.

Here, for the mathematical expression (1) (PA=a×A^{b}) describing the line 60 (depth: 5 mm) shown in FIG. 2, the coefficient “a” is 0.1137; the coefficient “b” is 0.7332. For the mathematical expression (1) describing the line 61 (depth: 10 mm), the coefficient “a” is 0.0618; the coefficient “b” is 0.7643. For the mathematical expression (1) describing the line 62 (depth: 15 mm), the coefficient “a” is 0.0424; the coefficient “b” is 0.7317. For the mathematical expression (1) describing the line 63 (depth: 20 mm), the coefficient “a” is 0.0365; the coefficient “b” is 0.6712. Thus, the coefficient “a” in the mathematical expression (1) decreases as the depth increases.

The PDT-index calculator 46 calculates or obtains the distribution of the PDT-index in the ROI based on the power of the last irradiation (here, the first irradiation) of the CW laser 13 for PDT detected by the power and waveform detector 15b and the concentration distribution of the photosensitizer at the specific depth D reobtained after the last irradiation of the CW laser 13 for PDT. The PDT-index represents the therapeutic effect of irradiating the tumor site with the therapeutic light or the PDT (photodynamic therapy). A mathematical expression (3) describes the therapeutic effect of the PDT (PDT-index). A PDT-index per unit time is obtained using a mathematical expression (4). The distribution of the obtained PDT-index is displayed on the monitor 42 in the form of an image.

(concentration of photosensitizer)×(power of irradiated CW laser for PDT)×(irradiation time)=const (3)

PDT-index=(concentration of photosensitizer)×(power of irradiated CW laser for PDT) (4)

The signal intensity of the ultrasonic signal 20 for the pulse laser for PAI (photo acoustic imaging) is proportionate to the PDT-index. “const(constant)” in the mathematical expression (3) is determined by the photosensitizer used (drug), a target site, a symptom, a type of the patient, and the like.

Using the following mathematical expression (5), the irradiation time calculator 47 calculates or obtains, based on the PDT-index in the ROI, the irradiation time of the CW laser for PDT required for the therapy. The obtained distribution of the irradiation time is displayed on the monitor 42.

irradiation time=(1/PDT-index)×const (5)

Based on the concentration distribution of the photosensitizer obtained by the photosensitizer concentration calculator 45, the irradiation condition setting section 48 sets or changes the irradiation condition of the CW laser 13 for PDT. To set or change the irradiation condition, for example, the irradiation time may be extended or shortened and the optical power may be increased or reduced at one or more measurement positions from among multiple measurement positions located at the specific depth.

Here, the irradiation condition setting section 48 sets or changes the irradiation condition of the CW laser 13 for PDT such that the concentrations of the photosensitizer in the ROI reduce at different speeds or rates in accordance with the concentration distribution of the photosensitizer. For example, the CW laser 13 for PDT is irradiated to a portion having the photosensitizer in high concentration such that the speed in reducing the concentration increases at a predetermined or a constant rate. On the other hand, the CW laser 13 for PDT is irradiated to a portion having the photosensitizer in low concentration such that the speed in reducing the concentration decreases at a predetermined or a constant rate.

For example, when the portion having the photosensitizer in high concentration is surrounded by not heat-resistant portions, the irradiation of the CW laser 13 for PDT should not be too strong. On the other hand, when a portion having the photosensitizer in low concentration is located deep inside the tumor site, it is necessary to irradiate the portion with rather strong CW laser 13 for PDT. In such cases, to irradiate a portion having the photosensitizer in high concentration, the irradiation condition setting section 48 sets an irradiation condition such that the speed in reducing the concentration decreases at a predetermined or a constant rate. To irradiate a portion having the photosensitizer in low concentration, on the other hand, the irradiation condition is set such that the speed in reducing the concentration increases at a predetermined or a constant rate.

For example, at the depth D (20 mm) in the tumor site, when an amplitude value PA_{P1} of the ultrasonic signal 20 at the measurement position P1 is half the amplitude value PA_{P2} of the ultrasonic signal 20 at the measurement position P2 different from the measurement position P1, namely, PA_{P1}=0.5×PA_{P2}, the irradiation condition is changed as follows. When the depth D is 20 mm, the relation between the amplitude value of the ultrasonic signal 20 and the absorbance is represented by the mathematical expression PA=0.0365×A^{0.6712} as described above. Accordingly, the amplitude value PA_{P1} at the measurement position P1 is represented by the following mathematical expression (6). The amplitude value PA_{P2} at the measurement position P2 is represented by the following mathematical expression (7).

PA_{P1}=0.0365×A_{P1}^{0.6712} (6)

PA_{P2}=0.0365×A_{P2}^{0.6712} (7)

A mathematical expression (8) is obtained from the above mathematical expressions (6) and (7).

PA_{P1}/PA_{P2}=(A_{P1}/A_{P2})^{0.6712} (8)

PA_{P1}/PA_{P2} is 0.5, so a mathematical expression (9) is obtained.

0.5=(A_{P1}/A_{P2})^{0.6712} (9)

The mathematical expression (9) is rewritten, with respect to A_{P1}, as a mathematical expression (10).

A_{P1}=A_{P2}(0.5)^{1.4899}=0.356×A_{P2} (10)

Thus, the absorbance A_{P1} at the measurement position P1 is 0.356 times as high as the absorbance A_{P2} at the measurement position P2. In view of the mathematical expression (3) representing the PDT effect, when the CW laser 13 for PDT is irradiated with the constant power, the irradiation condition of the CW laser 13 for PDT is changed to make the irradiation time for the measurement position P1 2.8 times longer than the irradiation time for the measurement position P2.

For example, at the depth D (20 mm) in the tumor site, when the concentration is reduced by half at the time t_{d} before the estimated time tₑₓ, the remaining irradiation time is extended 2.8 times longer than the predetermined or estimated remaining irradiation time. Accordingly, when the irradiation time is extended as described above, the total irradiation time t′ₑₓ is represented by a mathematical expression (11).

t′ₑₓ=t_{d}+(tₑₓ−t_{d})×2.8 (11)

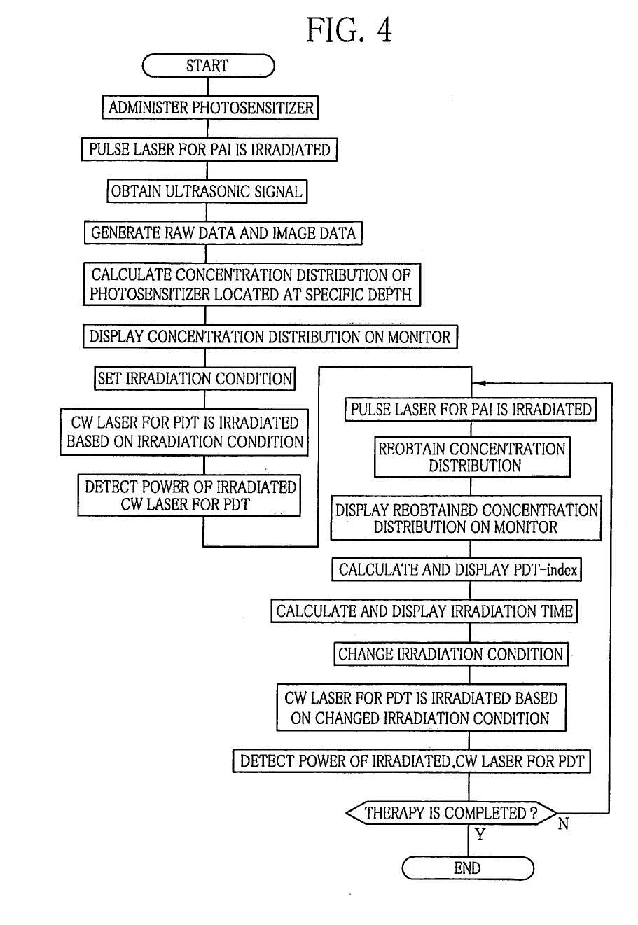

Next, referring to a flow chart in FIG. 4, an operation of the present invention is described. First, the photosensitizer is administered to the patient. Thereby, the photosensitizer accumulates in the tumor site of the patient. Then, the pulse laser 14 for PAI is irradiated from the laser source 15 to the tumor site in the living tissue 11. The photosensitizer in the tumor site absorbs the pulse laser 14 for PAI (photo acoustic imaging), and thereby the ultrasonic waves 17 are emitted from the tumor site in the living tissue 11. The ultrasonic wave detection device 18 detects the ultrasonic waves 17 to output the ultrasonic signal 20. The outputted ultrasonic signal 20 is sent to the processor device 22. During the irradiation of the pulse laser 14 for PAI, the power and waveform detector 15b detects the power and the waveform of the pulse laser 14 for PAI to output the power and waveform data 25. The outputted power and waveform data 25 is sent to the processor device 22.

The RAW data generator 30 in the processor device 22 generates one frame of RAW data based on the power and waveform data 25 of the pulse laser 14 for PAI and the ultrasonic signal 20. The signal processor 32 performs various processes such as the noise reduction process to the RAW data. Thereafter, based on the processed RAW data, the image data generator 34 generates image data of the ultrasonic tomographic image of the tumor site in the living tissue 11. Based on the image data, the monitor 42 displays the ultrasonic tomographic image of the tumor site in the living tissue 11.

Based on the image data generated by the image data generator 34, the photosensitizer concentration calculator 45 calculates or obtains the concentration distribution of the photosensitizer at the specific depth D in the tumor site. To be more specific, the concentration distribution of the photosensitizer in the ROI (region of interest) which is inputted by the operator using the ROI selector 55 is obtained. The concentration distribution of the photosensitizer in the ROI is obtained based on the amplitude values PA_{P1} to PA_{Pn} of the ultrasonic signals 20 detected at the measurement positions P1 to Pn located at the specific depth D and optical path differences Lₚ₁ to Lₚₙ between the measurement positions P1 to Pn and the exit section of the laser source 15.

After the concentration distribution of the photosensitizer at the specific depth D in the tumor site is obtained, the monitor 42 displays the concentration distribution in the form of an image. The irradiation condition setting section 48 sets the irradiation condition of the CW (continuous wave) laser 13 for PDT (photodynamic therapy) based on the concentration distribution of the photosensitizer. Based on the irradiation condition, the laser controller 49 generates the trigger signal 52. Based on the trigger signal 52, the laser driver 16 controls the laser source 15.

In accordance with the irradiation condition, the laser source 15 irradiates the tumor site in the living tissue 11 with the CW laser 13 for PDT. Thereby, the CW laser 13 for PDT is irradiated to a portion in the tumor site having the photosensitizer in high concentration so as to expedite the reduction in concentration. On the other hand, the CW laser 13 for PDT is irradiated to a portion in the tumor site having the photosensitizer in low concentration so as to slowdown or delay the reduction in concentration. Here, the power and waveform detector 15b detects the power and the waveform of the irradiated CW laser 13 for PDT to output the power and waveform data. The outputted power and waveform data is sent to the processor device 22.

After the CW laser 13 for PDT has been irradiated to the tumor site in the living tissue 11 for the predetermined time, the pulse laser 14 for PAI is irradiated to the tumor site in the living tissue 11. Then, the concentration distribution of the photosensitizer at the specific depth is reobtained in the same manner as above. The image representing the concentration distribution of the photosensitizer and displayed on the monitor 42 is updated based on the reobtained concentration distribution of the photosensitizer.

Based on the power of the last irradiation of the CW laser 13 for PDT (here, the first irradiation of the CW laser 13 for PDT) detected by the power and waveform detector 15b and the reobtained concentration distribution of the photosensitizer, the PDT-index calculator 46 calculates or obtains the PDT-index of the ROI. The monitor 42 displays the PDT-index, allowing the operator or doctor to check the progress of the treatment in the form of a numerical expression.

When the PDT-index is obtained, the irradiation time calculator 47 calculates the irradiation time of the CW laser 13 for PDT based on the PDT-index in the ROI. The monitor 42 displays the irradiation time, allowing the operator or doctor to check the irradiation time during the therapy.

Based on the reobtained concentration distribution of the photosensitizer, the irradiation condition setting section 48 changes the irradiation condition of the CW laser 13 for PDT in the same manner as the above. Based on the changed irradiation condition, the CW laser 13 for PDT is irradiated to the tumor site in the living tissue 11.

As described above, every time the CW laser for PDT is irradiated, the concentration of the photosensitizer is reobtained. Then, the CW laser for PDT is irradiated again based on the irradiation condition set or changed according to the reobtained concentration. Every time the concentration of the photosensitizer is reobtained, the concentration distribution of the photosensitizer, the PDT-index, and the irradiation time are displayed on the monitor 42. Thereby, changes in the concentration of the photosensitizer at a specific depth in the tumor site and the progress of the therapy, displayed as color fading on the monitor 42, are monitored. Thus, the safe and reliable therapy is achieved. The PDT is completed when the concentration of the photosensitizer at each of the measurement positions reaches zero or close to zero. Thus, risk and cost caused by excessive therapy are prevented.

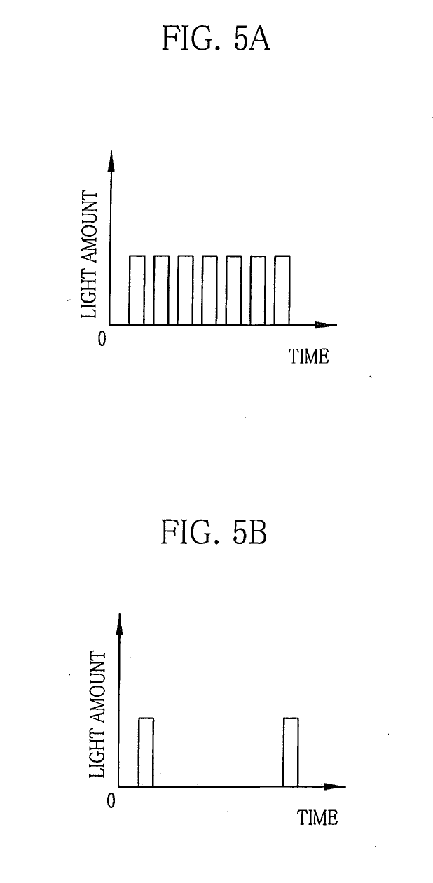

In this embodiment, the CW laser is used as the laser for the PDT. Alternatively, the pulse laser may be used as the laser for the PDT. In this case, the pulse laser for PDT may be used as the laser for PAI when one of the pulses of the pulse laser for PDT satisfies the conditions required for the laser for PAI, for example, a pulse width and pulse power. For the PDT, the pulse laser for PDT shown in FIG. 5A is irradiated to the tumor site. For obtaining the concentration of the photosensitizer, a part of the pulse laser for PDT is irradiated to the tumor site as the laser for PAI as shown in FIG. 5B.

In this embodiment, the CW laser for PDT and the pulse laser for PAI are irradiated from the same light source. Alternatively, the CW laser for PDT and the pulse laser for PAI may be irradiated from different light sources.

The present invention is applicable to, for example, electronic endoscope systems and surgical microscope systems using the PDT. The present invention is applicable to any medical apparatus system using the PDT.

Various changes and modifications are possible in the present invention and may be understood to be within the present invention.

What is claimed is:

1. A medical apparatus for photodynamic therapy comprising:
a light source for irradiating concentration measurement light and therapeutic light to a tumor site, the concentration measurement light being used for obtaining concentration of a photosensitizer permeated into the tumor site, the therapeutic light being used for treating the tumor site, wherein a light amount of each of the concentration measurement light and the therapeutic light is adjustable;
an ultrasonic wave detection device for detecting ultrasonic waves to output an ultrasonic signal, the ultrasonic waves being generated by the photosensitizer absorbing the concentration measurement light;
a concentration distribution calculator for obtaining concentration distribution of the photosensitizer at a specific depth in the tumor site based on the ultrasonic signal; and
a therapeutic light controller for controlling an irradiation condition of the therapeutic light to be irradiated to the tumor site based on the concentration distribution.

2. The medical apparatus of claim 1, wherein a portion having signal intensity equal to or higher than a predetermined value in the tumor site is set as a region of interest, and the irradiation condition of the therapeutic light is controlled such that a speed in reducing the concentration differs according to the concentration distribution.

3. The medical apparatus of claim 2, wherein the irradiation condition is controlled such that the speed in reducing the concentration increases at a predetermined rate in a portion having the photosensitizer in high concentration in the region of interest, and the speed in reducing the concentration decreases at a predetermined rate in a portion having the photosensitizer in low concentration in the region of interest.

4. The medical apparatus of claim 2, wherein the irradiation condition is controlled such that the speed in reducing the concentration decreases at a predetermined rate in a portion having the photosensitizer in high concentration in the region of interest, and the speed in reducing the concentration increases at a predetermined rate in a portion having the photosensitizer in low concentration in the region of interest.

5. The medical apparatus of claim 1, further including a PDT-index calculator for obtaining a PDT-index based on the concentration distribution of the photosensitizer at the specific depth obtained after last irradiation of the therapeutic light and power of the irradiated therapeutic light, the PDT-index representing a therapeutic effect of irradiating the tumor site with the therapeutic light.

6. The medical apparatus of claim 5, further including an irradiation time calculator for calculating, based on the PDT-index, an irradiation time of the therapeutic light required for completion of therapy of the tumor site.

7. The medical apparatus of claim 1, wherein every time the concentration distribution is obtained, the obtained concentration distribution is displayed in a form of an image on a monitor.

8. The medical apparatus of claim 1, further including a distribution matching section for matching the irradiation distribution of the concentration measurement light to the irradiation distribution of the therapeutic light.

9. The medical apparatus of claim 1, wherein the therapeutic light is continuously irradiated in a predetermined light amount from the light source.

10. The medical apparatus of claim 1, wherein the therapeutic light is irradiated from the light source while the light amount is changed.

11. The medical apparatus of claim 10, wherein a part of the therapeutic light is used as the concentration measurement light.

12. A method for controlling therapeutic light comprising the steps of:
(A) irradiating a tumor site with concentration measurement light for obtaining concentration of a photosensitizer permeated into the tumor site, a light amount of the concentration measurement light being adjustable;
(B) detecting ultrasonic waves to output an ultrasonic signal, the ultrasonic waves being generated by the photosensitizer absorbing the concentration measurement light;
(C) obtaining a concentration distribution of the photosensitizer at a specific depth in the tumor site based on the ultrasonic signal; and
(D) controlling an irradiation condition of the therapeutic light based on the concentration distribution.

13. The method of claim 12, wherein in the step (D), the irradiation condition of the therapeutic light is controlled such that a speed in reducing the concentration differs according to the concentration distribution.

14. The method of claim 12, further including the steps of:
(E) detecting power of the therapeutic light irradiated based on the irradiation condition;
(F) repeating steps (A) to (C) after the irradiation of the therapeutic light to obtain an updated concentration distribution of the photosensitizer at the specific depth in the tumor site;
(G) obtaining a PDT-index based on the updated concentration distribution and the detected power of the therapeutic light, the PDT index representing a therapeutic effect of irradiating the tumor site with the therapeutic light.
